(19) United States
(12) Patent Application Publication
(10) Pub. No.: US 2025/0338406 A1
(43) Pub. Date: Oct. 30, 2025

(54) VENTILATOR WITH DISPLAY

(71) Applicant: Loewenstein Medical Technology S.A., Luxembourg (LU)

(72) Inventor: Fritz MEHR, Merenberg (DE)

(21) Appl. No.: 19/184,138

(22) Filed: Apr. 21, 2025

(30) Foreign Application Priority Data
Apr. 25, 2024 (DE) 10 2024 111 636.6

(51) Int. Cl. H05K 5/00; A61M 16/08; H05K 5/02; H05K 5/13

(52) U.S. Cl. CPC H05K 5/0018; A61M 16/0875; H05K 5/0204; H05K 5/13; A61M 2205/502

(57) ABSTRACT

A ventilator comprises a main housing, a display unit having a display and a display rear wall, a display housing which is formed shell-like having a shell opening for inserting the display unit; and a display holder, which mechanically couples the display housing with the main housing. The display housing has a reinforcing structure connected to an inner wall of the display housing in its interior. The reinforcing structure comprises multiple screw connections arranged in a particular way.

### CROSS-REFERENCE TO RELATED APPLICATIONS

The present application claims priority under 35 U.S.C. § 119 of German Patent Application No. 10 2024 111 636.6, filed Apr. 25, 2024, the entire disclosure of which is expressly incorporated by reference herein.

### BACKGROUND OF THE INVENTION

### 1. Field of the Invention

The invention relates to a ventilator having a display.

### 2. Discussion of Background Information

A ventilator can comprise a main housing for accommodating components required for ventilation operation and a display fastened on the main housing for displaying ventilation-relevant information. Since the display is often touched by various persons in operation, regular cleaning of the display is necessary for hygienic reasons. The display is therefore to have the smoothest possible, rounded outer surface so that no dirt and/or no liquid can collect on irregularities such as steps, edges, scratches, or holes when the outer surface is wiped off.

In view of the foregoing, it would be advantageous to have available a ventilator having a display, the outer surface of which can be cleaned efficiently.

### SUMMARY OF THE INVENTION

The present invention provides a ventilator which comprises: a main housing for accommodating components which are required for ventilation operation of the ventilator, wherein the main housing comprises a respiratory gas fitting for connecting a respiratory gas source and/or a tubing system leading to a patient; a display unit having a display for displaying ventilation-relevant information and a display rear wall, which comprises a base plate fastened on a rear side of the display and multiple first screw connection sections protruding from the base plate; a display housing, which is formed shell-like having a shell opening for inserting the display unit, wherein the display unit is accommodated by the display housing so that the display at least partially covers the shell opening with its front side opposite to the rear side; a display holder, which mechanically couples the display housing with the main housing. In its interior, the display housing has a reinforcing structure connected to an inner wall of the display housing for reinforcing the display housing. The reinforcing structure comprises multiple second screw connection sections which are arranged so that they are each opposite to at least one of the first screw connection sections in an overlap area when the display unit is completely inserted into the display housing. The first and the second screw connection sections are screwed together by means of at least one screw element in each of the overlap areas, so that the display unit is fixed on the display housing. Each of the screw elements is arranged completely in the interior of the display housing here. Multiple access openings are formed in an outer wall of the display housing so that each of the screw elements is accessible for a screwing tool via one of the access openings from outside the display housing.

Due to the shell-like form of the display unit and the internal screw connection points, it is possible to prevent excess dirt or liquid from collecting at specific points when the display unit is wiped off. This can advantageously be achieved with the aid of a particularly simple and nonetheless robust structure of the ventilator, in particular of the display housing and/or the display unit.

The ventilator can be designed for invasive and/or noninvasive ventilation of a patient.

The respiratory gas fitting can comprise multiple separate inlets and/or outlets, for example, a respiratory gas inlet and a respiratory gas outlet. It is particularly favorable if the respiratory gas fitting is partially or completely installed in a housing front of the main housing.

A “respiratory gas source” can be understood, for example, as a gas cylinder, a respiratory gas mixer, a respiratory gas line system (for example, in a hospital building), or a combination of at least two of these examples.

A “tubing system” can be understood as an arrangement made up of one or more pieces of tubing for fluidically connecting the ventilator to a patient interface, for example, a ventilation mask, a tube, or a nasal cannula.

A “display” can be understood, for example, as an LCD or an OLED display.

The display can also completely cover the shell opening with its front side. An entire front side (facing toward an observer of the display) of the display unit inserted into the display housing can be formed here by the front side of the display, in particular by a front side of a display pane made of glass and/or plastic.

A “base plate” can in general be understood as a plate-like or grid-like support structure for supporting the display. For example, the base plate can be manufactured from metal and/or plastic. The base plate can in particular be embodied as a sheet-metal part and/or welded part.

The first screw connection sections can each protrude diagonally or perpendicularly from the base plate. For example, the first screw connection sections can be manufactured at least partially by corresponding bending over of sections of the base plate. Additionally or alternatively, the first screw connection sections can at least partially be plate-shaped sections, for example, sheet-metal sections, connected to the base plate by material bonding, in particular by welding and/or soldering.

The shell-like display housing can be, for example, a housing or a housing part having a rounded outer surface. In other words, the display housing can be shaped so that its outer surface does not have any excessively sharp edges and/or any excessively pointy corners. In this way, undesired steps transitions on the outer surface can be avoided. The display housing can be manufactured from plastic and/or metal. For example, the display housing can be manufactured together with the reinforcing structure as a single part (for example, as a single injection-molded part) and/or from the same material (for example, from the same plastic material).

The display holder can be designed, for example, to couple the display housing movably with the main housing so that the display housing can be adjusted in its position and/or orientation relative to the main housing and can be fixed in the adjusted position and/or orientation. For example, the display holder can comprise a special friction mechanism for the (for example, continuous) adjustment of a friction between the display housing and the main housing. The friction can preferably be adjustable by means of the friction mechanism so that the display housing can be moved with a certain application of force manually into a desired position and/or orientation and when it is released remains in the desired position and/or orientation solely due to the friction generated by the friction mechanism.

The display holder can expediently hold the display housing in relation to an upper side of the main housing in the operationally-ready state of the ventilator. However, a lateral arrangement of the display housing is also possible,

The reinforcing structure can comprise, for example, a plurality of reinforcing ribs connected to the inner wall of the display housing. The reinforcing ribs can be aligned parallel and/or orthogonal to one another. In other words, the reinforcing ribs can comprise longitudinal and/or transverse ribs. Additionally or alternatively, an oblique alignment of the reinforcing ribs in relation to one another is possible. The reinforcing ribs can extend at least partially curved and/or straight viewed in their respective longitudinal direction. For example, the reinforcing ribs can be bent at least partially in the form of a circle, circular segment, or ring.

For example, the first and the second screw connection sections can each be opposite to one another in pairs in an overlap area. In other words, each of the overlap areas can comprise precisely one of the first screw connection sections and precisely one of the second screw connection sections in each case. However, overlap areas having more than two screw connection sections are also possible, for example, having at least one first screw connection section and at least two second screw connection sections or having at least two first screw connection sections and at least one second screw connection section. For example, in the first case the first screw connection section can be arranged between the second screw connection sections and/or in the second case the second screw connection section can be arranged between the first screw connection sections.

A “screw element” can be understood, for example, as a screw, a threaded bolt, a screw nut, or a combination of at least two of these examples.

It is possible that each of the access openings is arranged at a defined distance to an end of the respective screw element facing toward the access opening. This enables the screw elements to be arranged particularly deep in the interior of the display housing, so that the screw elements cannot be seen from the outside or can only be seen when they are looked for more closely.

To enable particularly stable fixing of the display unit, the ventilator can comprise at least four different overlap areas.

Various embodiments of the invention are described hereinafter. These embodiments are not to be understood as a restriction of the scope of the invention.

According to one embodiment, a distance of each one of the access openings to an end of the respective screw element facing toward the access opening can be at least about 1 cm, at least about 2 cm, at least about 5 cm, or at least about 10 cm.

According to one embodiment, the screw elements can comprise multiple screws, wherein each of the screws is guided through a hole in one of the first screw connection sections and/or through a hole in one of the second screw connection sections and/or is screwed together with a screw nut which is arranged on one of the first screw connection sections and/or on one of the second screw connection sections. For example, the end of the screw element facing toward the access opening can be a screw head of the respective screw.

According to one embodiment, the display housing can comprise a shell bottom and a shell side wall connected to the shell bottom. The shell opening can be delimited by a circumferential edge (facing away from the shell bottom) of the shell side wall. Furthermore, the display housing can be delimited in the direction of its height (in the operational state of the ventilator), on the one hand, by an upper section of the shell side wall and, on the other hand, by a lower section of the shell side wall. Additionally or alternatively, the display housing can be delimited in the direction of its width, i.e. in a direction transverse to its height (in the operational state of the ventilator), on the one hand, by a left section of the shell side wall and, on the other hand, by a right section of the shell side wall. The access openings can be formed here in at least one of the sections of the shell side wall. Alternatively, it is possible that at least one of the access openings is formed in the shell bottom.

Additionally or alternatively, the display housing can be delimited in the direction of its depth, i.e. in a direction transverse to its height and width (in the operational state of the ventilator), on the one hand, by the shell bottom and, on the other hand, by the display, in particular by the display rear wall.

According to one embodiment, the access openings can be formed outside the upper section of the shell side wall. This can prevent dirt or liquid from reaching the interior of the display housing during cleaning of the display housing through the access openings due to gravity.

According to one embodiment, the access openings can only be formed in one of the sections of the shell side wall, in particular only in its lower section. The screw elements therefore only have to be screwed in or detached on one side of the display housing in order to install or remove the display unit. This simplifies the maintenance, restoration, and repair of the display unit in comparison to an embodiment in which the screw elements are arranged on various sides of the shell side wall. If the access openings are only formed in the lower section of the shell side wall, this has the advantage that in the operational state of the ventilator the access openings are not recognizable or are only recognizable when looked for very closely from the outside, which contributes to a more aesthetic appearance of the ventilator. Moreover, in this way dirt or liquid is effectively prevented from reaching the interior of the display housing through the access openings during cleaning of the display housing.

According to one embodiment, the reinforcing structure can comprise a plurality of elongated reinforcing ribs. In this case, the reinforcing ribs can each be connected, on the one hand, to the shell side wall and, on the other hand, to the shell bottom. In other words, the reinforcing ribs can be designed to support the shell side wall on the shell bottom (and vice versa).

According to one embodiment, the reinforcing ribs can comprise a plurality of first reinforcing ribs arranged parallel to one another and a plurality of second reinforcing ribs arranged parallel to one another. In this case, the first reinforcing ribs can each be aligned orthogonally and/or obliquely to the second reinforcing ribs viewed in their longitudinal direction. In other words, the longitudinal direction of each one of the first reinforcing ribs can extend orthogonally or obliquely to the longitudinal direction of each one of the second reinforcing ribs. The reinforcing ribs can additionally comprise a plurality of further reinforcing ribs arranged parallel to one another, which can each be aligned orthogonally and/or obliquely to the first reinforcing ribs and/or the second reinforcing ribs viewed in their longitudinal direction. As mentioned above, the reinforcing ribs can extend at least partially curved and/or straight viewed in their respective longitudinal direction. For example, the reinforcing ribs can be at least partially bent in the form of a circle, circular segment, or ring.

According to one embodiment, the first reinforcing ribs can each be connected, on the one hand, to the upper section and/or the lower section of the shell side wall and, on the other hand, to the shell bottom. The first reinforcing ribs can each extend here over a part of the height of the display housing or over its entire height viewed in their longitudinal direction.

According to one embodiment, the second reinforcing ribs can each be connected, on the one hand, to the left section and/or the right section of the shell side wall and, on the other hand, to the shell bottom. The second reinforcing ribs can each extend here over a part of the width of the display housing or over its entire width viewed in their longitudinal direction.

According to one embodiment, at least one of the first reinforcing ribs can additionally be connected to at least one of the second reinforcing ribs. This can further enhance the stability of the display housing.

For example, at least one of the first reinforcing ribs can be connected only to the upper section of the shell side wall and the shell bottom (and additionally to at least one of the second reinforcing ribs) and at least one further one of the first reinforcing ribs can be connected only to the lower section of the shell side wall and the shell bottom (and additionally to at least one of the second reinforcing ribs).

Additionally or alternatively, at least one of the second reinforcing ribs can be connected only to the left section of the shell side wall and the shell bottom (and additionally to at least one of the first reinforcing ribs) and at least one further one of the second reinforcing ribs can be connected only to the right section of the shell side wall and the shell bottom (and additionally to at least one of the first reinforcing ribs).

According to one embodiment, a respective length of the reinforcing ribs can be at most about 30%, at most about 50%, or at most about 70% of the height and/or width of the display housing.

According to one embodiment, the reinforcing ribs can be at least partially of differing lengths and/or at least partially equal in length. For example, the first reinforcing ribs can at least partially differ from one another and/or can at least partially differ from the second reinforcing ribs in their lengths. Additionally or alternatively, the second reinforcing ribs can at least partially differ from one another in their length.

According to one embodiment, the display housing can have in its interior a display receptacle area, which is free of the reinforcing ribs, for accommodating at least one section of the display unit. This enables a particularly flat and nonetheless stable design of the display housing. A base area of the display receptacle area can be, for example, from about 20% to about 70%, in particular from about 20% to about 50% of a base area of the shell bottom.

According to one embodiment, at least one or each of the second screw connection sections can be a (for example, plate-like or sheet-like) section of one of the reinforcing ribs.

According to one embodiment, multiple shafts for guiding the screwing tool can be formed in the interior of the display housing. Each of the shafts can extend starting from one of the access openings toward the end of the respective screw element facing toward the access opening and/or toward one of the overlap areas. In this case, a shaft wall of each one of the shafts can be formed so that a screw element located in the shaft cannot fall out of the shaft into the interior of the display housing, for example, during the installation or removal of the display unit. For example, the shaft wall can be closed for this purpose. A non-enclosed shaft wall having correspondingly small openings is also possible, for example, a shaft wall formed at least partially like a grid or net. A “shaft” can be understood, for example, as a tubular or grooved element. It is expedient if the shaft extends linearly or only slightly curved viewed in its longitudinal direction in order to enable the insertion of an elongated screwing tool, for example, a blade of a screwdriver. For example, the shaft wall can comprise at least one of the following sections: a section of the shell bottom, a section of the shell side wall, a section of the reinforcing structure (in particular a reinforcing rib), a section of the display rear wall (in particular the base plate). This embodiment enables the screwing tool to be guided blind via the respective access opening to one of the screw elements. This can significantly simplify the installation or removal.

For example, the shaft wall can comprise sections of at least two first or second reinforcing ribs arranged parallel to one another.

According to one embodiment, the display can have a display pane made of glass and/or plastic on its front side. In this case, the display pane can largely or completely cover the shell opening. For example, the display pane can terminate flush with a circumferential edge of the shell opening viewed in the direction of the height and/or width of the display housing. The display unit can furthermore comprise an operating element, which is mounted so it is rotatable and/or displaceable relative to the display pane on and/or in a pane hole in the display pane. A “pane hole” can be understood as a passage opening in the material of the display pane delimited by a circumferential edge. In other words, the pane hole can connect a front side of the display pane to its rear side. The pane hole can be, for example, circular, oval, or elongated (i.e. an elongated hole).

According to one embodiment, the operating element can be fixed by means of an additional screw element on a first end of an elongated coupling element, wherein a second end of the coupling element can be mounted so it is rotatable and/or displaceable on and/or in the display housing. The additional screw element can be arranged completely in the interior of the display housing and an additional access opening can be formed in the outer wall of the display housing so that the additional screw element, in a specific position and/or orientation of the operating element relative to the display pane, is accessible for a screwing tool via the additional access opening from outside the display housing. A distance of the additional access opening to an end of the additional screw element facing toward the additional access opening can be at least about 1 cm, at least about 2 cm, at least about 5 cm, or at least about 10 cm. A “coupling element” can be understood as a rod-shaped element such as a bolt, a pin, or a tube. It is possible that the operating element protrudes through the pane hole and/or the coupling element protrudes through the pane hole. The additional access opening can be formed, for example, in one of the sections of the shell side wall, in particular in its lower section. The operating element can also be fixed by means of multiple additional screw elements at the first end of the coupling element. Multiple additional access openings can also be formed in the outer wall of the display housing.

According to one embodiment, the first end of the coupling element can be inserted into a recess of the operating element and fixed by means of an additional screw element in the recess. A “recess” can be understood, for example, as a cup-shaped or socket-shaped section of the operating element, in particular a cylindrical base body of the operating element, as is described hereinafter. The first end of the coupling element can engage in the recess in a formfitting manner, for example, such that slipping of the coupling element inserted into the recess relative to the recess is not possible around and/or along its center axis with normal application of force. In this case, an additional holding force can be exerted on the coupling element by means of the additional screw element.

According to one embodiment, the operating element can comprise a cylindrical base body and a collar protruding from an outer lateral surface of the base body having an outer contour (for example, annular) matched to an inner contour (for example, annular) of the pane hole. In this case, the base body can be guided through the pane hole so that it protrudes on both sides from the display pane. Moreover, the collar can be inserted into the pane hole so that the outer contour is at least partially surrounded by the inner contour. The collar can additionally be formed as a stop for fixing the base body (or the operating element) in at least one direction parallel to a center axis of the pane hole. The collar can extend partially or completely around the outer lateral surface of the base body in its circumferential direction.

According to one embodiment, the collar can terminate flush with the front side of the display. The front side of the display can be formed in particular by a (planar) outer surface of the display pane. In this case, the collar can terminate flush with the outer surface of the display pane bordering the pane hole, so that a transition between the collar and the outer surface of the display pane is essentially continuous. This can prevent dirt or liquid from collecting at the transition during cleaning of the display pane.

According to one embodiment, a gap (for example, annular) between the collar and the display pane, in particular between the outer contour of the collar and the inner contour of the pane hole, can be closed fluid-tight. For this purpose, the gap can be dimensioned correspondingly small and/or can be filled with a suitable sealing material. This prevents the penetration of dirt or liquid into the interior of the display unit and/or the display housing.

According to one embodiment, the collar can touch the display pane, in particular an edge section of the display pane bordering the pane hole, at an annular contact surface.

According to one embodiment, the inner contour of the pane hole and the outer contour of the collar can touch at an annular contact surface.

This prevents the penetration of dirt or liquid into the interior of the display unit and/or the display housing without additional sealing material having to be introduced into any gap between the collar and the display pane or between the inner contour and the outer contour.

According to one embodiment, the additional screw element can be inserted (for example, screwed) into a passage opening, which connects the outer lateral surface of the base body to an inner lateral surface of the base body and/or to an inner lateral surface of the recess, in particular far enough that the additional screw element protrudes beyond the inner lateral surface of the base body into the interior of the base body or beyond the interior lateral surface of the recess into the interior of the recess. One end of the additional screw element can press here with a suitable force in a direction transverse or diagonal to the center axis of the base body or the recess against the first end of the coupling element in order to fix it in the base body or in the recess.

According to one embodiment, the display can be electrically conductively connected via a detachable plug connection for power supply and/or for data communication to at least one of the components in the main housing. The plug connection can be detached, for example, by merely removing the display holder from the main housing and/or from the display housing. Vice versa, it is possible that the plug connection is established solely in that the display holder is inserted up to a defined end position into the main housing and/or into the display housing. The plug connection can be embodied, for example, as a USB or USB-C plug connection and/or can be arranged completely in the interior of the ventilator, for example, at least partially in the interior of the main housing and/or at least partially in the interior of the display holder and/or at least partially in the interior of the display housing. This enables the display to be removed quickly and easily if needed, for example, to operate it somewhere other than directly at the ventilator.

According to one embodiment, the ventilator can furthermore comprise a light source for targeted illumination of at least one section of the ventilator and/or the surroundings of the ventilator. A “light source” can be understood, for example, as an incandescent bulb, a light-emitting diode, or a combination of at least two of these examples. The light source can be controllable in its brightness and/or color manually or automatically, for example, depending on the measured brightness in the surroundings of the ventilator. In the simplest case, the light source can be able to be switched on or off manually by means of a suitable switch. The light source can be installed, for example, in an outer wall of the main housing, in particular in a housing front of the main housing.

According to one embodiment, in addition to the display (as a main display), the ventilator can have an additional display for displaying ventilation-relevant information. The additional display can be installed, for example, in an outer wall of the main housing, in particular in a housing front of the main housing. Accordingly, the additional display can have a significantly smaller display area than the main display. The ventilator can be designed to additionally display information required for ventilation operation on the additional display. This enables the ventilation operation to be maintained even if the main display is not available for some reason, for example, because it is impaired in its function due to a disturbance or is exchanged in running operation.

### BRIEF DESCRIPTION OF THE DRAWINGS

Embodiments of the invention are described hereinafter with reference to the appended drawings. Neither the description nor the drawings are to be understood as a restriction of the scope of the invention. In the drawings:

FIG. 1 shows a ventilator according to one embodiment of the invention.

FIG. 2 shows details of a display housing of a ventilator according to one embodiment of the invention.

FIG. 3 shows a cross section through the display housing from FIG. 2 with inserted display unit along a section line A-A.

FIG. 4 shows a cross section through a section, having an operating element, of a display unit of a ventilator according to one embodiment of the invention.

The drawings are solely schematic and are not to scale. If identical reference signs are used in different drawings, these reference signs designate identical or identically-acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

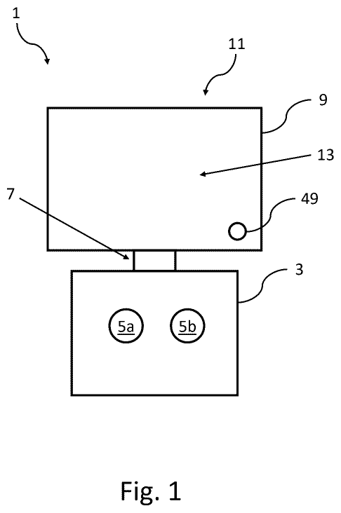

FIG. 1 shows a ventilator 1 for invasive and/or noninvasive ventilation of a patient.

The ventilator 1 comprises a main housing 3, which encloses components required for a ventilation operation of the ventilator 1, for example, one or more components of a controller, a sensor system, and/or an actuator system of the ventilator 1. In this example, the main housing 3 has a respiratory gas fitting on its housing front, which can comprise a respiratory gas inlet 5a for connecting a respiratory gas source and a respiratory gas outlet 5b for connecting a tubing system leading to the patient.

The main housing 3 is mechanically coupled via a display holder 7 with a display housing 9, in particular such that a user can adjust the display housing 9 in its position and/or orientation relative to the main housing 3 and can fix it in the respective position and/or orientation.

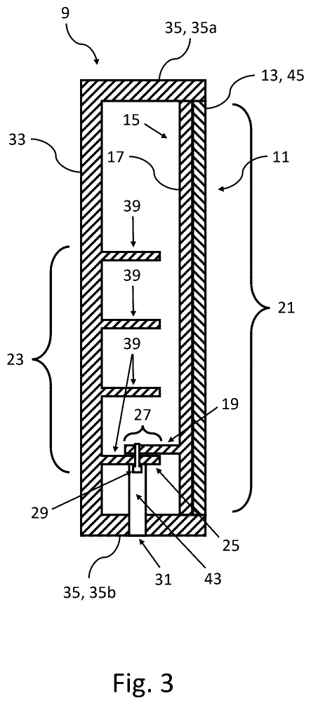

In addition, the ventilator 1 comprises a display unit 11 having a display 13 for displaying ventilation-relevant information and a display rear wall 15 (see FIG. 3), which comprises a base plate 17 fastened on a rear side of the display 13 and multiple first screw connection sections 19 protruding from the base plate 17.

The display housing 9 is formed shell-like having a shell opening 21. The display unit 11 is accommodated by the display housing 9 so that the display 13 partially, largely, or completely covers the shell opening 21 with its front side, which is opposite to the rear side.

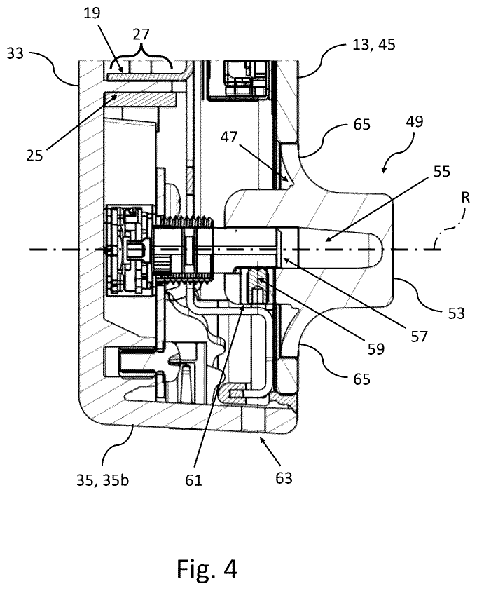

The display housing 9 has in its interior a reinforcing structure 23 connected to an inner wall of the display housing 9 for reinforcing the display housing 9. The reinforcing structure 23 comprises multiple second screw connection sections 25, which are arranged so that they are each opposite to at least one of the first screw connection sections 19 in an overlap area 27 when the display unit 11—as shown in FIG. 3 and FIG. 4—is completely inserted into the display housing 9.

The first screw connection sections 19 and the second screw connection sections 25 are screwed together in each of the overlap areas 27 by means of at least one screw element 29, in the form of a screw here, so that the display unit 11 is fixed on the display housing 9 and is therefore adjustable together with the display housing 9. Each of the screw elements 29 is located completely in the interior of the display housing 9 here.

Furthermore, multiple access openings 31 are formed in an outer wall of the display housing 9 so that each of the screw elements 29 is accessible via one of the access openings 39 from outside the display housing 9 for a screwing tool, for example, a screwdriver. A distance D of each one of the access openings 31 to an end of the respective screw element 29 facing toward the access opening 31 can be, for example, at least 1 cm, at least 2 cm, at least 5 cm, or at least 10 cm.

In this example, each of the screw elements 29 is guided in the respective overlap area 27 through a hole in one of the first screw connection sections 19 and through a further hole in one of the second screw connection sections 25 and is screwed together with a screw nut (not shown), for example, in the form of a press-in nut, on the respective second screw connection section 25.

The display housing 9 can be delimited by a shell bottom 33 and a shell side wall 35, which is connected to the shell bottom 33 and extends around it. The shell opening 21 can be delimited here by a circumferential edge of the shell side wall 35 facing away from the shell bottom 33.

Accordingly, the display housing 9 can be delimited in the direction of its height H, on the one hand, by an upper section 35a of the shell side wall 35 and, on the other hand, by a lower section 35b of the shell side wall 35 and also in the direction of its width B, on the one hand, by a left section 35c of the shell side wall 35 and, on the other hand, by a right section 35d of the shell side wall 35.

All access openings 31 can be formed in at least one of the four sections of the shell side wall 35. In this example, all four access openings 31 are formed in the lower section 35b.

The reinforcing structure 23 can comprise a plurality of elongated reinforcing ribs, which can each be connected, on the one hand, to the shell side wall 35 and, on the other hand, to the shell bottom 33.

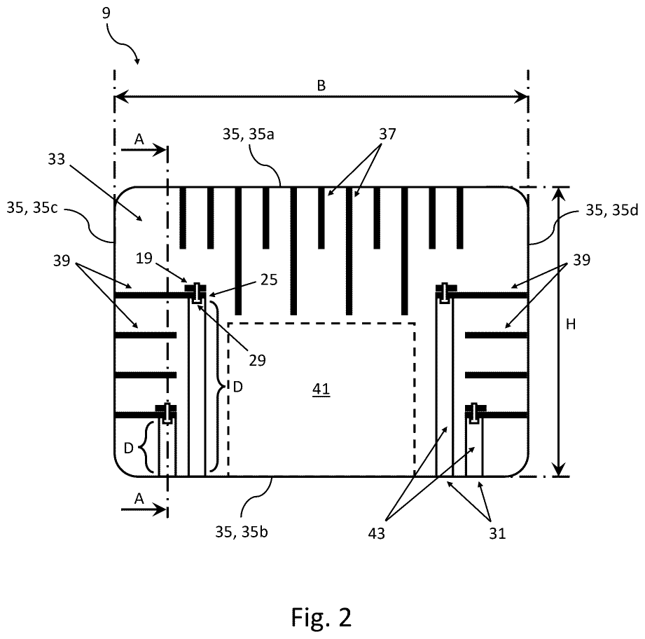

In this example, the reinforcing ribs comprise a plurality of first reinforcing ribs 37 arranged parallel to one another and a plurality of second reinforcing ribs 39 arranged parallel to one another, which differ in their respective longitudinal direction from the first reinforcing ribs 37. For example, the first reinforcing ribs 37, as shown in FIG. 2, can each be aligned orthogonally to the second reinforcing ribs 39 viewed in their longitudinal direction. Alternatively, an oblique alignment is possible.

The first reinforcing ribs 37 are each connected, on the one hand, to the upper section 35a and, on the other hand, to the shell bottom 33. Some of the second reinforcing ribs 39 are each connected, on the one hand, to the left section 35c and, on the other hand, to the shell bottom 33, wherein the remaining second reinforcing ribs 39 are each connected, on the one hand, to the right section 35d and, on the other hand, to the shell bottom 33. However, other connections are also possible. For example, the reinforcing ribs 37, 39 can also be connected to one another and/or can interconnect sections of the shell side wall 35 opposite to one another.

As can be seen in FIG. 2, the respective length of the reinforcing ribs 37, 39 is significantly less than 50% of the height H or the width B, so that a specific area of the display housing 9, more precisely the shell bottom 33, is free of reinforcing ribs. This omitted area can function as a display receptacle area 41, by which a specific rear-side section of the display unit 11 is accommodated when it is completely inserted into the display housing 9. This enables a particularly flat design of the display housing 9.

In this example, the total of four second screw connection sections 25 are different sections of four of the second reinforcing ribs 39, more precisely of two left and two right second reinforcing ribs 39.

In addition, multiple shafts 43 for guiding the screwing tool can be formed in the interior of the display housing 9. In this example, the shafts 43 each extend linearly in the direction of the height H starting from one of the access openings 31 toward one of the screw elements 29, more precisely toward an end of one of the screw elements 29 facing toward the respective access opening 31, here a screw head.

A shaft wall of each one of the shafts 43 is expediently formed so that a screw element 29 located in the shaft 43 cannot inadvertently fall out of the shaft 43 into the interior of the display housing 9 during the installation or removal of the display unit 11.

The shaft wall can comprise a section of the shell bottom 33 and/or sections opposite to one another of two of the reinforcing ribs 37 or 39 aligned parallel to one another.

Additionally or alternatively, the shaft wall can comprise a section of the display rear wall 15, for example, the base plate 17, and/or a section of the shell side wall 35.

Depending on the embodiment, the display 13 can have a display pane 45 made of glass and/or plastic on its front side. The display pane 45 can largely or completely cover the shell opening 21.

As can be seen in FIG. 4, the display pane 45 can have a pane hole 47 delimited by a (completely) circumferential edge, which connects a front side of the display pane 45 to its rear side. An operating element 49 for operating the display 13 and/or the ventilator 1 can be mounted so it is rotatable and/or displaceable relative to the display pane 45 in and/or on the pane hole 47.

In this example, the operating element 49 is embodied as a rotating knob rotatable relative to the display pane 45 around an axis of rotation R. The operating element 49 can comprise a cylindrical base body 53 having a cup-shaped or socket-shaped recess 55 here. The axis of rotation R corresponds here by way of example with the center axis of the base body 53. The base body 53 can be guided through the pane hole 47, which is circular, for example, so that viewed in the direction of the axis of rotation R, it protrudes on both sides from the display pane 45, i.e. both from its front side and from its rear side. A first end of a rod-shaped coupling element 57 can be inserted into the recess 55 and fixed therein by means of an additional screw element 59. In contrast, a second end of the coupling element 57 can be mounted so it is rotatable around the axis of rotation R in the interior of the display housing 9, for example, on the shell bottom 33.

The additional screw element 59, for example, a grub screw, is screwed here in a direction transverse to the axis of rotation R into a passage opening 61, which connects an outer lateral surface of the base body 53 to an inner lateral surface of the recess 55.

Moreover, an additional access opening 63 can be formed in the outer wall of the display housing 9 so that the additional screw element 59 is accessible for a screwing tool in a specific rotational position of the operating element 49 (or the base body 53) via the additional access opening 63 from outside the display housing 9.

In the example shown in FIG. 4, the additional access opening 63 is formed like the other access openings 31 in the lower section 35b of the shell side wall 35. Accordingly, the additional screw element 59 is accessible for the screwing tool when the operating element 49 is rotated into a position in which the additional screw element 59 is vertically opposite with one of its ends to the additional access opening 63, i.e. when the additional access opening 63 and the additional screw element 59 lie one on top of another, in particular such that the center axis of the additional access opening 63 and the longitudinal axis of the additional screw element 59 lie on a common straight line or on straight lines parallel to one another with a correspondingly small distance from one another. In this position, a distance of the additional access opening 63 to an end of the additional screw element 59 facing toward the additional access opening 63 can be, for example, at least 1 cm, at least 2 cm, at least 5 cm, or at least 10 cm.

The operating element 49 can additionally comprise a collar 65, which protrudes from an outer lateral surface of the base body 53 and annularly encloses it in its circumferential direction. The collar 65 can have an annular outer contour matched to an annular inner contour of the pane hole 47 and can be inserted into the pane hole 47 such that its outer contour is at least partially enclosed by the inner contour of the pane hole 47. The inner contour and outer contour can be adapted to one another in their diameter such that they are separated from one another by an air gap (for example, likewise annular). The air gap can additionally be closed fluid-tight using a suitable sealing material. It is alternatively possible that the inner contour and outer contour touch at an annular contact surface, in particular such that no water can penetrate into the interior of the display housing 9 via the contact surface. Moreover, the collar 65 can be formed as an axial stop for positioning the base body 53 in the direction of the axis of rotation R relative to the display pane 45.

To prevent dirt or liquid from collecting at the transition between the collar 65 and the display pane 45, the collar 65 can be shaped so that-viewed in the direction of the axis of rotation R—it terminates flush with a front side of the display pane 45 facing toward an observer of the display 13, i.e. the transition can be formed essentially continuous.

Finally, it is to be noted that terms such as “has”, “comprises”, “includes”, “having”, etc. do not exclude other elements or steps and indefinite articles such as “a” or “an” do not exclude a plurality.

Furthermore, it is to be noted that features or steps which are described with reference to one of the preceding embodiments can also be used in combination with features or steps which are described with reference to others of the preceding embodiments.

Reference signs in the claims are not to be understood as a restriction of the scope of the subject matter defined by the claims.

### LIST OF REFERENCE NUMERALS

| | |
|---|---|
| 1 | ventilator |
| 3 | main housing |
| 5a | respiratory gas inlet |
| 5b | respiratory gas outlet |
| 7 | display holder |
| 9 | display housing |
| 11 | display unit |
| 13 | display |
| 15 | display rear wall |
| 17 | base plate |
| 19 | first screw connection section |
| 21 | shell opening |
| 23 | reinforcing structure |
| 25 | second screw connection section |
| 27 | overlap area |
| 29 | screw element |
| 31 | access opening |
| 33 | shell bottom |
| 35 | shell side wall |
| 35a | upper section |
| 35b | lower section |
| 35c | left section |
| 35d | right section |
| 37 | first reinforcing rib |
| 39 | second reinforcing rib |
| 41 | display receptacle area |
| 43 | shaft |
| 45 | display pane |
| 47 | pane hole |
| 49 | operating element |
| 53 | base body |
| 55 | recess |
| 57 | coupling element |
| 59 | additional screw element |
| 61 | passage opening |
| 63 | additional access opening |
| 65 | collar |
| B | width |
| D | distance |
| H | height |
| R | axis of rotation |

What is claimed is:

1. A ventilator, wherein the ventilator comprises:
a main housing for accommodating components required for a ventilation operation of the ventilator, the main housing comprising a respiratory gas fitting for connecting a respiratory gas source and/or a tubing system leading to a patient;
a display unit having a display for displaying ventilation-relevant information and a display rear wall, which comprises a base plate fastened on a rear side of the display and multiple first screw connection sections protruding from the base plate;
a display housing, which is formed shell-like and has a shell opening for inserting the display unit, the display unit being accommodated by the display housing so that the display at least partially covers the shell opening with its front side opposite to the rear side;
a display holder, which mechanically couples the display housing with the main housing;
wherein the display housing has in its interior a reinforcing structure connected to an inner wall of the display housing for reinforcing the display housing, wherein the reinforcing structure comprises multiple second screw connection sections, the second screw connection sections being arranged so that they are each opposite to at least one of the first screw connection sections in an overlap area when the display unit is completely inserted into the display housing, wherein the first screw connection sections and the second screw connection sections are screwed together in each of the overlap areas by at least one screw element, so that the display unit is fixed on the display housing, wherein each of the screw elements is arranged completely in the interior of the display housing, wherein multiple access openings are formed in an outer wall of the display housing so that each of the screw elements is accessible for a screwing tool via one of the access openings from outside the display housing.

2. The ventilator of claim 1, wherein a distance (D) of each one of the access openings to an end of the respective screw element facing toward the access opening is at least 1 cm.

3. The ventilator of claim 1, wherein a distance (D) of each one of the access openings to an end of the respective screw element facing toward the access opening is at least at least 2 cm.

4. The ventilator of claim 1, wherein a distance (D) of each one of the access openings to an end of the respective screw element facing toward the access opening is at least 5 cm.

5. The ventilator of claim 1, wherein the screw elements comprise multiple screws, each of the multiple screws being guided through a hole in one of the first screw connection sections and/or through a hole in one of the second screw connection sections and/or is screwed together with a screw nut, which is arranged on one of the first screw connection sections and/or on one of the second screw connection sections.

6. The ventilator of claim 1, wherein the display housing comprises a shell bottom and a shell side wall connected to the shell bottom, the shell opening being delimited by a circumferential edge of the shell side wall, wherein the display housing is delimited in a direction of its height (H), on the one hand, by an upper section of the shell side wall and, on the other hand, by a lower section of the shell side wall and/or in a direction of its width (B), on the one hand, by a left section of the shell side wall and, on the other hand, by a right section of the shell side wall, the access openings being formed in at least one of the sections of the shell side wall.

7. The ventilator of claim 6, wherein the access openings are formed outside the upper section; and/or are only formed in one of the sections of the shell side wall.

8. The ventilator of claim 6, wherein the access openings are formed in a lower section of the shell side wall.

9. The ventilator of claim 6, wherein the reinforcing structure comprises a plurality of elongated reinforcing ribs, each of them being connected, on the one hand, to the shell side wall and, on the other hand, to the shell bottom, wherein the reinforcing ribs comprise a plurality of first reinforcing ribs arranged parallel to one another and a plurality of second reinforcing ribs arranged parallel to one another, the first reinforcing ribs, viewed in their longitudinal direction, each being aligned orthogonally and/or obliquely to the second reinforcing ribs.

10. The ventilator of claim 9, wherein the first reinforcing ribs are each connected, on the one hand, to the upper section and/or the lower section of the shell side wall and, on the other hand, to the shell bottom; and/or
wherein the second reinforcing ribs are each connected, on the one hand, to the left section and/or the right section of the shell side wall and, on the other hand, to the shell bottom; and/or
wherein at least one of the first reinforcing ribs is additionally connected to at least one of the second reinforcing ribs.

11. The ventilator of claim 9, wherein a respective length of the reinforcing ribs is at most 30% of the height (H) and/or the width (B) of the display housing; and/or
wherein the display housing has a display receptacle area free of the reinforcing ribs in its interior for accommodating at least one section of the display unit.

12. The ventilator of claim 9, wherein at least one or each of the second screw connection sections is a section of one of the reinforcing ribs.

13. The ventilator of claim 1, wherein multiple shafts for guiding the screwing tool are formed in the interior of the display housing, wherein each of the shafts extends starting from one of the access openings toward an end of the respective screw element facing toward the access opening, wherein a shaft wall of each one of the shafts is formed so that a screw element located in the shaft cannot fall out of the shaft into an interior of the display housing.

14. The ventilator of claim 13, wherein the shaft wall comprises a section of at least one of the reinforcing ribs.

15. The ventilator of claim 1, wherein the display has a display pane made of glass and/or plastic on its front side, the display pane largely or completely covering the shell opening;
and wherein the display unit furthermore comprises an operating element, which is mounted so it is rotatable and/or displaceable relative to the display pane on and/or in a pane hole in the display pane.

16. The ventilator of claim 15, wherein the operating element is fixed by an additional screw element at a first end of an elongated coupling element and a second end of the coupling element is mounted to be rotatable and/or displaceable on and/or in the display housing, wherein the additional screw element is arranged completely in the interior of the display housing, wherein an additional access opening is formed in an outer wall of the display housing so that the additional screw element is accessible via the additional access opening from outside the display housing for a screwing tool in a specific position and/or orientation of the operating element relative to the display pane.

17. The ventilator of claim 16, wherein the first end of the coupling element is inserted into a recess of the operating element and is fixed by the additional screw element in the recess; and/or
wherein the operating element comprises a cylindrical base body and a collar protruding from an outer lateral surface of the base body having an outer contour matched to an inner contour of the pane hole, wherein the base body is guided through the pane hole, so that it protrudes on both sides from the display pane, wherein a collar is inserted into the pane hole so that the outer contour is at least partially enclosed by the inner contour.

18. The ventilator of claim 17, wherein the collar terminates flush with the front side of the display; and/or
wherein a gap between the collar and the display pane is closed fluid-tight; and/or
wherein the collar and the display pane touch at an annular contact surface; and/or
wherein the inner contour and outer contour touch at an annular contact surface; and/or
wherein the additional screw element is inserted into a passage opening, which connects the outer lateral surface of the base body to an inner lateral surface of the base body and/or to an inner lateral surface of a recess.
